(19) United States
(12) Patent Application Publication
(10) Pub. No.: US 2008/0133002 A1
(43) Pub. Date: Jun. 5, 2008

(54) Inflatable artificial valve

(76) Inventors: Daniel Gelbart, Vancouver (CA); Samuel Victor Lichtenstein, Vancouver (CA)

Correspondence Address: DANIEL GELBART, 4706 DRUMMOND DR, VANCOUVER, BC, V5, omitted

(21) Appl. No.: 11/633,447

(22) Filed: Dec. 5, 2006

(51) Int. Cl. A61F 2/24; A61F 2/02

(52) U.S. Cl. 623/2.1; 623/23.68

(57) ABSTRACT

An artificial valve is delivered percutaneously in the form of a hollow inflatable shell, which is later filled in situ with a suitable elastomer, similar to inflating a balloon. Since the shell is a flexible and mostly hollow balloon, it can be compressed and transported via a relatively small catheter. In order to inflate to the correct shape the hollow shell requires some ties between its walls. These ties are formed from a low density open cell foam or a specialized manufacturing process. The valve can combine elements with different stiffness as well as different materials. A positioning balloon can also be used in the process of placing the valve. The invention is particularly suited for cardiac valves but can be used for other artificial valves.

### FIELD OF INVENTION

The invention relates to artificial valves installed in the body percutaneously, and in particular to cardiac valves.

### BACKGROUND OF THE INVENTION

Artificial valves, and in particular artificial cardiac valves, have been used for a long time. Recently the trend is to install them percutaneously, via a catheter. Current catheters capable of delivering today's valve are fairly large (size 24Fr and sometimes larger). This greatly limits the use of such valves as the hole left in the vascular system or the heart wall is significant. An object of the invention is to deliver a large size valve, such as a cardiac valve, via a very small catheter without damaging the valve in the process. A second object is to have a valve than can be anchored in an irregular area, such as to replace a faulty mitral valve. A third object is to seat the valve forming a hemostatic seal without any high local pressures that can cause calcified deposits to break off, such as in an aortic valve. Mqre objects and advantages will become apparent from studying the full disclosure and the drawings.

### SUMMARY OF THE INVENTION

According to the invention, the artificial valve is delivered percutaneously in the form of a hollow inflatable shell, which is later filled in situ with a suitable elastomer, similar to inflating a balloon. Since the shell is a flexible and mostly hollow balloon, it can be compressed and transported via a relatively small catheter. In order to inflate to the correct shape the hollow shell requires some ties between its walls. These ties are formed from a low density open cell foam or via a manufacturing process detailed in the disclosure. The valve can combine elements with different stiffness as well as different materials. A positioning balloon can also be used in the process of placing the valve. The invention is particularly suited for cardiac valves but can be used for other artificial valves.

### BRIEF DESCRIPTION OF THE DRAWINGS

FIG. 1 is a cross section of a human heart showing catheter delivery of an artificial mitral valve.

FIG. 2 is a general view of the valve inside a delivery catheter (made of transparent material for clarity).

FIG. 3-A shows the deployed valve before inflation.

FIG. 3-B shows the deployed valve after inflation.

FIG. 4 is a cross section of the heart showing the valve inflated inside the mitral valve annulus.

FIG. 5-A is an isometric view of a bi-cuspid embodiment.

FIG. 5-B is a cross section of a bi-cuspid embodiment.

FIG. 6-A is an isometric view of a tri-cuspid embodiment.

FIG. 6-B is a cross section of a tri-cuspid embodiment.

FIGS. 7-A, 7-B and 7-C show, in cross section, the mold and method used for manufacturing the valve.

FIGS. 8-A, 8-B, 8-C and 8-D show, in cross section, an alternate manufacturing method.

FIGS. 9-A, 9-B, 9-C show a “lost wax” manufacturing method.

### DESCRIPTION OF THE PREFERRED EMBODIMENT

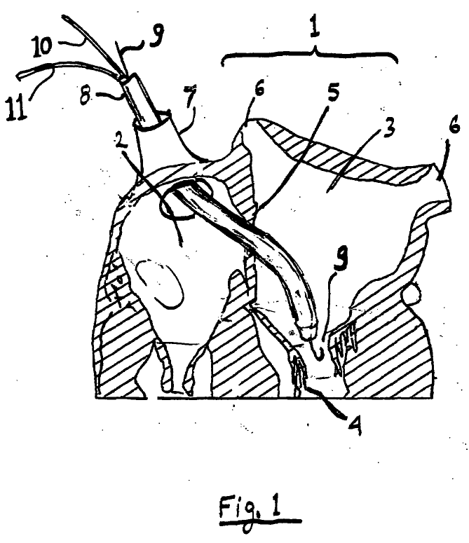

While the invention is suitable for many different artificial valves in the human body, the example given in the preferred embodiment will be a mitral valve replacement. FIG. 1 is a partial cross section of a human heart 1, comprising of a right atrium 2, left atrium 3, mitral valve 4, septum 5, pulmonary veins 6 and superior vena cava 7. A catheter 8 is introduced percutaneously via the superior vena cava 7 into the left atrium 3 via the septum 5. Normally a guide wire 9 is introduced first, followed by a catheter having a dilator (not shown). After catheter 8 is in place, the valve is introduced. The valve is connected to the outside of the body by two inflation tubes, 10 and 11 (details of the valve insertion are given later). In this example the catheter is positioned near the mitral valve. The procedure of percutaneously delivery is well known to those skilled in the art of interventional cardiology or catheter based surgical procedures. It is similar to the well known procedure of placing stents inside the arterial system.

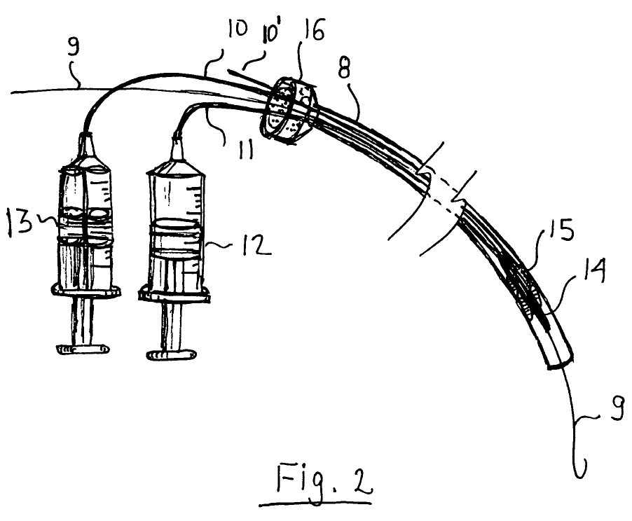

FIG. 2 is a more detailed view of the catheter. Catheter 8 is drawn as if it was transparent for greater clarity. Catheter 8 has a hemostatic seal 16 on the outside end allowing the insertion and removal of tools and devices without loss of blood. Typically the seal is made of soft foam. Inside catheter 8 is guide wire 9, positioning balloon 14 connected to tube 11 and inflatable valve 15 connected to tube 10. Each inflation tube is connected to an inflation device, such as a syringe. Syringe 11 contains a saline solution for inflating balloon 14 while syringe 13 contains typically an elastomer that will solidify once injected into inflatable artificial valve 15. In order to allow a long working time while placing the device, and a short curing time for the elastomer once injected into the valve, a two-part elastomer is desired. Most suitable elastomers, such as silicones and polyurethanes, are supplied as two-part systems, becoming a flexible solid a short time after mixing. For such applications syringe 13 is divided into two compartments, each one carrying one part of the elastomer, and the two parts mixing together in the delivery tube 10 (or in a special mixing nozzle, not shown). The art of supplying elastomer and other adhesives in a two-part syringe is well known as many popular adhesives, such as “5 minute Epoxy” are sold in this form. An alternative is to use a thermoplastic elastomer, supplied at a slightly elevated temperature such as 50 to 70 deg C. It is desirable to equip syringes 12 and 13 with pressure gages (not shown) in order not to exceed the pressure rating of the balloon 14 and valve 15. Such use of pressure gages is well known in the art of angioplasty. It may be desirable to also supply an overflow tube 10′ connected to the inflatable valve to allow any trapped air to escape and to verify complete filling. The amount of trapped air is minimal, as the valve is fully compressed inside catheter 8. Clearly the overflow tube 10′ needs to be sealed off before final pressure can be reached. As an alternative, compressed valve 15 and tube 10 can be pre-filed with one part of the elastomer while a single compartment syringe 13 delivers the other part. In many silicones only a small amount of catalyst is needed to cure a large volume, thus the catalyst can be in the compressed valve while the resin in syringe 13. This is the case with Dow Corning 3110 RTV silicone, where the catalyst volume is only a few percent of the liquid silicone rubber volume. The valve can be pre-filled with catalyst and the liquid silicone rubber can be injected by a single syringe. In yet another embodiment, the valve is self-inflating by the reaction of two components of a “foam-in-place” elastomer. The valve is pre-filled with a small amount of each component, separated by a thin partition. As the valve is expanded by the balloon the partition breaks and both components mix and inflate the valve. Since such elastomeric foams are capable of expanding 10× and even 100× their initial volume, a very small amount of liquid is needed inside the compressed valve

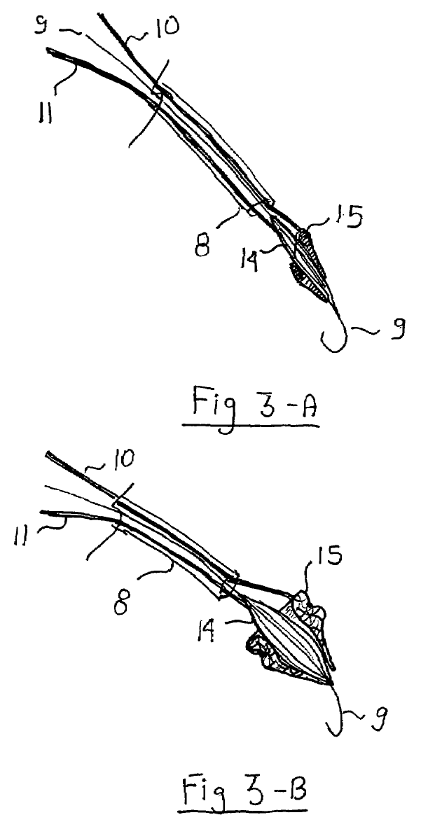

Once catheter 8 is in position, balloon 14 and valve 15 are pushed out as shown in FIG. 3-A. They can be pushed using tubes 10 and 11 or a separate flexible wire (not shown). Guide wire 9 guides the balloon 14 into place and balloon 14, once inflated, positions valve 15 in place as shown in FIG. 3-B. The invention can be practiced without a guide wire or balloon; the balloon helps position the valve as the valve is very soft and elastic and therefore difficult to grip, while balloon 14 and tube 11 can be more rigid, serving as a “handle” to manipulate valve into place. Since tube 10 has to be easily detachable from valve it is preferred to handle the valve indirectly, via balloon 14. Balloon 14 is similar in constructions to balloons currently used to place valves in cardiac surgery and need not be detailed here. At the end of the procedure, balloon 14 is deflated and removed through catheter 8. To further reduce catheter size, valve 15 can be placed in catheter behind balloon 14. After balloon is pushed out of catheter, valve can be pushed out, partially inflated and pushed over balloon. This allows a very small catheter, down to 16 Fr or even 12 Fr. Filling tube 10 (and overflow tube 10′, if used) is detached from valve by pulling or twisting and is removed as well. FIG. 4 shows the inflated valve in place, before removal of the balloon. Because the valve is inflatable and compliant is can form a hemostatic seal over irregular surfaces such as a calcified aortic valve or delicate surfaces such as a mitral valve annulus 4, even if the annulus is covered with plaque or scar tissue the valve will inflate to form a seal.

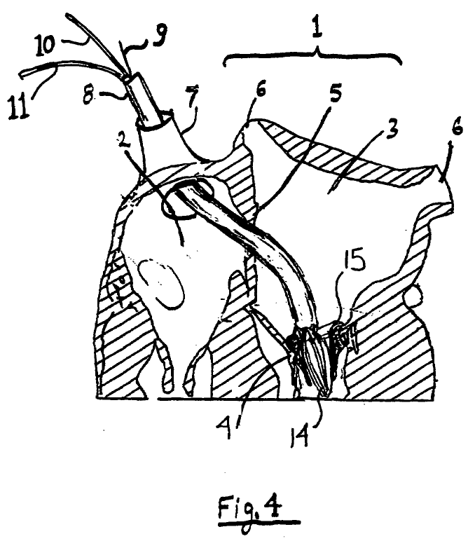

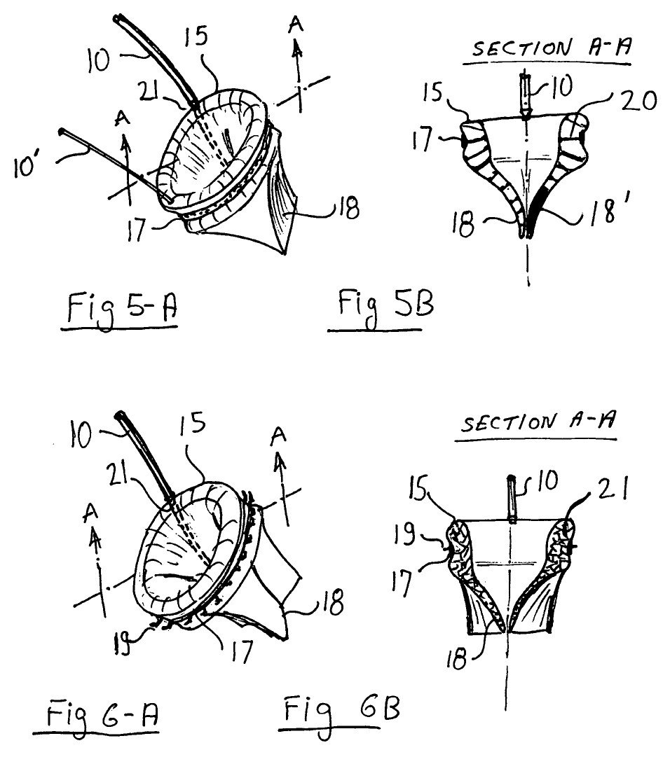

The valves can be modeled after natural valves, such as a bi-cuspid configuration shown in FIG. 5-A and 5-B, tri-cuspid, as shown in FIG. 6-A and 6-B, reed or disc valve (not shown) or any other configuration. In FIG. 5-B is a cross section of the valve shown in 5-A along the plane marked A-A. Similarly, FIG. 6-B is a cross section of 6-A. Valve 15 is formed by a hollow balloon in the shape of the valve with some internal reinforcement 20 which will be discussed later. The valve may contain a recessed band 17 made of a more rigid material order to prevent excessive shape distortion. The location of recessed band 17 is aligned with the annulus into which the valve fits. Once inflated, the recessed shape forms a lock preventing the valve from being pushed out. Since the pressure on the valve can be significant (about a kilogram in a mitral valve), band 17 needs to be significantly stiffer than the rest of the valve. Additional texturing of band 17 promotes adhesion and tissue growth. A Velour-like finish is known to greatly increase adhesion in cardiac valves and if desired, barbs 19 can be added as shown in FIG. 6. The barbs lock valve in place immediately, unlike tissue bonding that develops over time. When using barbs it is desired to place them at an angle pointing up (opposite direction to blood flow), not only to resist the upward force better, but to avoid puncturing the valve when compressed into the catheter, as valve is pushed through catheter in the direction causing barbs to fold.

The lower part of valve 15 forms multiple leaflets 18 that seal tighter the more blood pressure is applied. The leaflets need to be fairly thin (preferably 0.5-2 mm) and very flexible (preferably durometer of 20-40 Shore A scale) in order to minimize pressure drop in the forward direction. The properties of the non-flexing part of the valve are less critical. The materials for the valve are chosen not only by good elastomeric properties (essential for long fatigue life) but mainly by bio-compatibility, primarily minimizing blood clotting. From past experience both silicones and polyurethanes are suitable material. Materials can be further enhanced by special surface preparations such as drug eluting coating, carbon coatings, highly hydrophobic coatings, special textures promoting epithelium growth etc. The advantage of being able to grow epithelium over the valve is the minimizationreduction or elimination of anti-clotting medication. The inflatable shell need not be of the same material as the injected filler; a polyurethane shell can be filled with a silicone elastomer and vice versa. The filler need not be a solid: a silicone gel, silicone fluid, saline solution or water-based gel can be used. In the latter case the compressed valve can contain the dried gel powder which expands in volume when water is added. Elastomer filled valves are more durable and tear-resistant. The advantage of valves filled with a saline solution is that they can be removed by puncturing and draining the fluid. In some special cases there can be an advantage to fill the valve with a gas such as CO2. Polymers are somewhat permeable to gases, this can be used as an advantage when the valve is required to supply a higher pressure against the seating surface till it is held by tissue growth. Suitable bio-compatible fillers and fluids are well known in the art of plastic surgery and in particular breast and penile implants. While most of the valve is made of an inflatable shell, some parts can be made of solid material. Because leaflets 18 are thin they can be made of solid elastomer, as shown by 18′ in FIG. 5-B. Reinforcing ring 17, if required, can also be made of solid material which can be different from the valve material. For example, ring 17 can be a Nitinol wire. Filling tube 10 is detachably connected to valve 15. This can be done via a thinner section 21, which will break when tube 10 is pulled, or by making tube lo a separate part and inserting it into valve 15. After elastomer inside valve 15 solidified, tube 10 is detached. When valve is filled with a liquid or gel instead of elastomer, section 21 has to be a one-way filling valve. It can be as simple as a self-sealing minute opening in valve, opened by the pressure of the fluid in tube 10. Sometimes it may be desired to add a detachable overflow tube 10′ (in FIG. 5-A) to verify complete filling. Tube 10′ can be smaller than tube 10. By the way of example, for filling with polyurethane tube 10 should be 1-3 mm ID while tube 10′ can be slightly less. For filling with silicone rubber tube 10 should to be 1.5-3 mm ID. In general tubes should be made as large as possible, since the factor governing the catheter size is the diameter of the folded valve. The valve shown in FIG. 5 and FIG. 6 will not inflate properly without internal reinforcement 20. The reason is that any inflated shell will try to maximize the internal volume for a given surface area, which does not lead to the desired shape. Two types of internal reinforcements will be detailed, including three manufacturing method, but there is a large number of possible methods. The internal reinforcement can be in the form of thread-like braces 20 in FIG. 5-B or a low density open cell foam as shown by 21 in FIG. 6-B.

The amount the valve can be compressed depends primarily on the amount of solid material in the shell compared to the total volume. By the way of example, for a 25 mm diameter valve the wall thickness can be 0.1-0.5 mm. If a different, less elastomeric, material can be used for the outside shell the thickness of the shell can be greatly reduced (dramatically reducing the catheter size). By the way of example, if the shell can be made of polyimide (Kapton) a shell as thin as 0.03 mm can be used. The shell does not require the same long fatigue life the elastomer core requires: even if the shell cracks over time the valve will function properly.

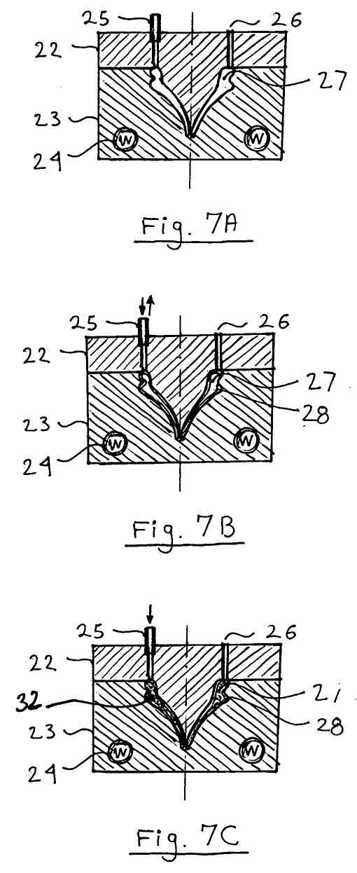

FIGS. 7, 8 and 9 depict three alternate manufacturing processes for the invention. FIG. 7 is a cross section of the mold used to make the valve. The mold is made of metal and comprises of a top part 22, bottom part 23, heaters 24, filling tube 25 and overflow tube 26. Cavity 27 is of the shape of the finished valve. At first a low viscosity elastomer (pre-mixed with catalyst) is injected via tube 25 and drained out (by inverting mold and using suction). A thin coating is left on the inside walls of cavity 27, as shown by shell 28 in FIG. 7-B. Because the mold is heated, curing time is typically minutes but can be as fast as a few seconds (only partial curing is needed). The thickness of shell 28 is determined by the viscosity of the liquid elastomer. Next a foam-in-place elastomer liquid is injected in the mold and quickly expands to fill the mold, as shown in FIG. 7-C. It is important that the foam will be of the open cell type, with a low density (about 80 to 95% air), as it needs to be filled with an elastomer or liquid once inside the body. Open cell foaming elastomers are well known in the art of polymers and are commercially available, for example under the trade names Icynene (www.icynene.com) and Sealite (www.insulstar.com). The elastomer used to form the shell and later on the filler are well known in the art. Silicones are available from DowCorning (www.dowcorning.com) and polyurethanes are available, for example, from Bayer (www.bayermaterialscience.com). After a short curing time finished valve can be removed by separating top part 22 from bottom 23. The examples given here are for industrial materials; clearly only the medically approved version of such materials can be used.

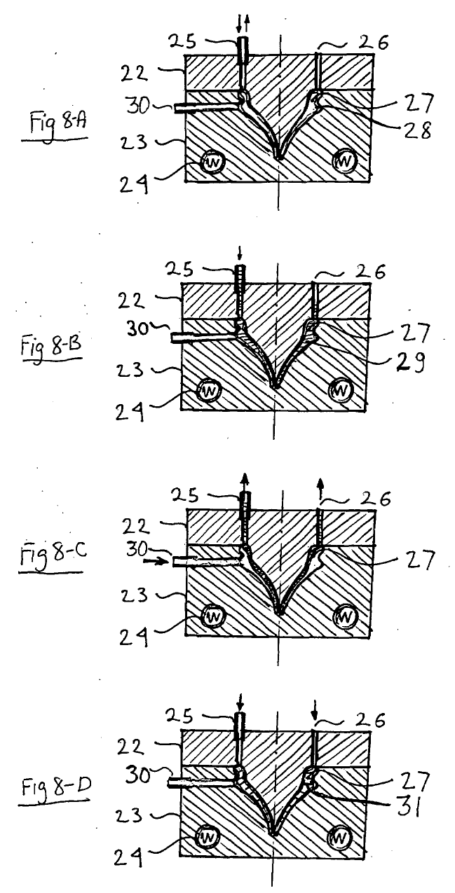

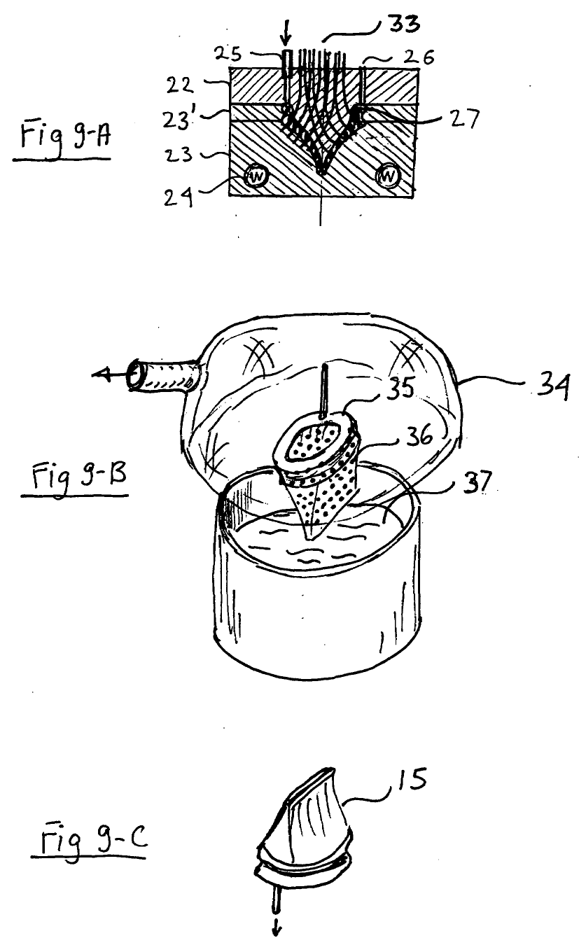

In order to generate the reinforcement shown in FIG. 5-B the method shown in FIG. 8 is used. After a shell 28 is formed in FIG. 8-A (using the same methods as in FIG. 7-A and 7-B) and cured, a higher viscosity elastomeric adhesive 29 is introduced as shown in FIG. 8-B. Immediately after step 8-B compressed air is introduced via tube 30, compressing the shell 28 as shown in FIG. 8-C and ejecting most of the elastomeric adhesive via tubes 25 and 26. Before any significant curing occurred, compressed air is applied via tubes 25 and 26, as shown in FIG. 8-D, expanding the shell again but leaving behind elastomeric bridges 31. The formation of the elastomeric bridges depends on the viscosity and tack of the elastomeric adhesive. These properties can be somewhat modified by waiting a short interval (seconds) between step 8-C and 8-D, as partial curing greatly increases the viscosity of elastomeric adhesive 29. Sometimes it is desirable to add a micro-fiber filler to adhesive 29 to get the desired filament formation (similar to the filament formed when trying to remove chewing gum). After step 8-D the valve is left in the heated mold till fully cured.

The exact formulations, curing times and mold temperatures greatly depend on the desired properties. While not required, it is easier to fabricate the shell and the elastomeric filler from the same polymer family, in order to generate a homogeneous structure. By the way of example, the shell and the filler can both be made from Dow Coming 3110 RTV with type 4 catalyst, mold heated to about 100 deg C. filaments 31 made from Dow Corning Adhesive/Sealant. With those materials a complete molding cycle takes a few minutes, as does the curing inside the body. Faster curing silicones and urethanes can be used to reduce both the manufacturing cycle as well as the curing cycle inside the body to under 1 minute.

A different process, based on the lost wax metal casting process, is shown in FIG. 9-A to FIG. 9-C. Using this process will require significantly longer time to make a valve, however, the process is more flexible and gives better control in case of complex valves. In this process a mold is made not in the shape of the final valve, but in the shape of the airspace inside the valve. The mold is shown in FIG. 9-A and is similar to the molds in the previous process except that heaters 24 are set to a low temperature or not used at all. In order to create the shapes of the reinforcement 31 (see FIG. 8-D) in the valve, wires 33 can be used. Hot wax is injected via port 25. When wax is solidified, wires 33 are pulled out and mold is opened. Because wax is not flexible, undercuts should be eliminated and mold may need to be separated into more segments, as shown by 23 and 23′ in FIG. 9-A. The wax core 35, representing the airspace in the valve, is shown in FIG. 9-B. The holes 36, left behind by wires 33, will form the reinforcements when filled with polymer. To create the inflatable shell and fill the holes 36, the wax core 35 is dipped in a catalyzed (or pre-mixed) elastomer solution 37 and withdrawn, leaving just a thin coat over the core 35. In order to assure the penetration of the elastomer into holes 36 it is recommended to do the process under vacuum, shown schematically as a vacuum jar 34. The art of vacuum impregnating a core with a polymer under vacuum is well known and is a standard process in encapsulation. The vacuum is released before the elastomer is cured, collapsing all small bubbles and forcing the liquid elastomer into all cavities of core 35. After core 35 is coated it is left to cure at room temperature or a low temperature (about 10 minutes for Dow Corning 3110 RTV). At the point it is heated to above the melting point of the wax (typically 100 deg C) and all the wax is removed, as shown in FIG. 9-C while still hot the inside of valve 15 is washed out repeatedly with a solvent of a high boiling point such as naphta (petroleum distillate). It is important to eliminate any trace of wax to ensure good bonding with the filler elastomer to be injected later.

AS in all molding processes attention has to be paid to mold release and wetting properties. When using silicones wetting and mold release are not a problem and normally no mold releasing agents are required. When using polyurethanes mold releases are normally required and wetting agents may be required to coat the inside of the mold or the wax core in an even layer.

1. An artificial valve delivered to its destination in the body in the form of an inflatable shell and inflated in place by a fluid.

2. A valve as in claim wherein said fluid polymerizes into an elastomer.

3. A valve as in claim 1 wherein said fluid turns into a gel.

4. A valve as in claim 1 wherein said fluid is a gas.

5. A valve as in claim 1 wherein said valve is a cardiac valve.

6. A valve as in claim 1 wherein said valve is delivered percutaneously.

7. A valve as in claim 1 wherein said valve is delivered percutaneously and positioned with the aid of an inflatable balloon.

8. A valve as in claim 1 wherein said inflatable shell and said fluid are of the same polymer family.

9. A valve as in claim 1 wherein different parts of said valve are made of different materials.

10. A valve as in claim 1 wherein said valve is pre-filled with a catalyst which reacts with said inflating fluid to form an elastomer.

11. A valve as in claim 1 wherein said inflatable shell has an internal reinforcing structure assisting in controlling the inflated shape.

12. A valve as in claim 1 wherein said valve is manufactured by a molding process.

13. A valve as in claim 1 wherein said valve is manufactured by a lost wax process.

14. An artificial valve having at least part of said valve inflatable by the injection of a fluid after said valve is delivered into the body.

15. An inflatable valve as in claim 1 wherein said inflating fluid is stored inside valve and inflates the valve by the creation of an elastomeric foam.

16. A method of installing an artificial valve in the human body comprising of the following steps:
percutaneously delivering an inflatable shell to the desired location; and
inflating said shell to the final valve form by injecting a fluid.

17. A method as in claim 12 wherein said valve is a cardiac valve.

18. A method as in claim 12 wherein said fluid polymerizes into an elastomer.
